(19) United States
(12) Patent Application Publication
(10) Pub. No.: US 2018/0280431 A1
(43) Pub. Date: Oct. 4, 2018

(54) Process for Obtaining the Product for Prevention, Interruption of Dental Caries Lesions and Teeth Remineralization and Obtained Product

(71) Applicant: MINISTERIO DA CIENCIA E TECNOLOGIA, Cidade Universitária, Recife (BR)

(72) Inventors: ANDRÉ GALEMBECK, Piedade, Jaboatão dos Guararapes (BR); MIGUEL ANGEL PELAGIO FLORES, Iputinga, Recife (BR); VALDECI ELIAS DOS SANTOS JUNIOR, Piedade, Jaboatão dos Guararapes (BR); FABIANA DE GODOY BENÉ BEZERRA LAUREANO, Boa Viagem, Recife (BR); ANDRÉA GADELHA RIBEIRO TARGINO, Manaira, João Pessoa (BR); ARONITA ROSENBLATT, Pina, Recif (BR); RODRIGO JOSÉ DE OLIVEIRA, Sandra Cavalcante, Campina Grande (BR); HILZETH DE LUNA FREIRE PESSÔA, Manaira, João Pessoa (BR)

(21) Appl. No.: 15/571,320

(22) PCT Filed: Jul. 16, 2015
(86) PCT No.: PCT/BR2015/050092

§ 371 Date: Nov. 2, 2017

(30) Foreign Application Priority Data
May 4, 2015 (BR) BR1020150100892

(51) Int. Cl. A61K 33/38; A61K 33/16; A61K 47/36; A61K 47/10; A61K 47/12; A61K 47/22; A61K 9/70; A61K 9/00; A61P 1/02

(52) U.S. Cl. CPC A61K 33/38; A61K 33/16; A61K 47/36; A61K 47/10; B82Y 5/00; A61K 47/22; A61K 9/7007; A61K 9/0053; A61P 1/02; A61K 47/12

(57) ABSTRACT

Addresses the invention of product comprising: metal particles and/or free or complexed metal ions; fluoride; organic acid and polysaccharides, with or without additives, designed to prevent the occurrence of tooth lesions caused by dental caries, interrupt the development of such lesions, and to promote the remineralization of the affected teeth and a process of obtaining this product.

### FIELD OF THE INVENTION

The present invention finds its scope among the products that were made to prevent the occurrence of lesions in teeth caused by dental caries, interrupt the development of such lesions, as well as promote the remineralization of the affected tooth. More specifically, a product containing a hybrid complex nanostructured material with metallic particles of nanometric dimensions, metal ions and fluorides encapsulated in polysaccharides. A process of obtaining this product is also presented.

### BACKGROUND OF THE INVENTION

It is estimated that over 80% of the world population have suffered or suffer from dental caries at some point in their lives.

The treatment for carious lesion, which is a condition of infection having the Streptococcus muttans as the primary pathogen, is carried out until today through the mechanical removal of carious tissues with drills. Recently and even more rarely, the use of lasers to remove affected tissues have been applied. These procedures are very invasive and were executed in cold blood in the past, being extremely painful depending on the extent of the lesion. Even with the advent of anesthesia, the procedure is still unpleasant for adults. The procedure is critical and often traumatic for children, leading to a rejection behavior for the rest of their lives.

Among the coadjuvants for the treatment of carious lesion, silver is known for its antibacterial activity since the times of ancient Greece and works from the late 19th century and early 20th century already indicated the use of cariostatics containing silver nitrate, in order to interrupt carious lesion.

The silver diammine fluoride (SDF), for example, is an effective agent against dental caries when applied every 12 months, with proven action in the prevention of coronal caries. Coronal caries is the most common type of dental lesion. It occurs in both children and adults dentition. Coronal caries can be found on chewing surfaces or between the teeth. The SDF compound is found for distribution in the form of aqueous solutions with concentrations ranging from 10 to 38%, and it is easy to apply.

However, due to the low viscosity of the product and the presence of silver ions (ionic silver) the application must be carried out carefully, because they cause the appearance of dark spots that remain for a few weeks if it comes into direct contact with the skin.

Another disadvantage of this product comes from the formation of silver oxide. The application of SDF on the affected area, as well as on the skin, causes the appearance of a pigmentation presenting a gradation that ranges from brown to dark brown when in contact with the porous tooth enamel. This coloration aspect is highly undesirable, which stigmatize the patient, since the dark color evidences the treatment of rotten teeth and considerably limits the acceptance of its use by dentists.

The use of ionic silver and silver particles of nanometer dimensions as antimicrobial agents and, more specifically, as bacteriostatic and bactericidal has been reported in scientific literature, and it has also been the object of requests for privilege of invention. There are reports about the use of silver ions in the formulations for oral hygiene combined with fluorine sources, sweeteners and other additives. The compositions described might be useful for preventing and treating dental caries and gingivitis according to the data obtained through in vitro studies.

metal particles of nanometric dimensions also present proven bacteriostatic and bactericidal action.

The nanometric silver has been used in various applications in several fields of technology to reduce infections in the treatment of injuries caused by burning, arthroplasty, to prevent bacterial colonization on prosthesis, catheters, vascular grafts, some dental restorative materials to eliminate microorganisms in textile fabrics and water treatment, due to its efficient bacteriostatic and antibacterial activity against S. muttans microorganisms.

The WO 2006074117 document reports that compositions containing nanometric silver in aqueous solutions and in the form of gel are used in various antimicrobial applications such as genital and urinary infections, skin infections caused by bacteria or fungi and infections in the respiratory system. The WO 2007017901 document describes compositions in the form of gel containing silver particles of nanometric dimensions with proven action against E. coli, P. aeruginosa and S. aureus, which can be applied in microbial infections in both skin and oral cavity. The WO_2010072882_A1 document presents materials in the form of calcium phosphate powder and nanometric silver for use as bactericidal and/or fungicidal, and proposes its application as disinfectant in: public areas, surgical implants, odontology in general, food, paint, clothing and packaging. This document, however, does not provide examples of specific application for any of these sectors.

Chitin is the second most abundant polysaccharide in nature, after cellulose and it is present in the exoskeleton of crustaceans, insects, mollusks and fungi. Its production comes from the shell of crustaceans such as lobster, crab and shrimp, which contain 15 to 25% of chitin.

Chitosan is a linear chain polysaccharide, cationic with protonation (addition of protons) of the amino group (NH3+), and it can be obtained through the deacetylation of chitin, differing from this in terms of proportion of amino groups and in terms of solubility.

Chitin has a proven antibacterial activity against a broad spectrum of bacteria and it has also shown important characteristics for the human cells such as biocompatibility, biodegradability, bio adhesion and non-toxicity. The antimicrobial activity of chitosan depends on its molecular weight and deacetylation degree.

The use of polysaccharide compounds, including chitosan, is due to the fact that this polymer is widely used in the medical field due to its low immune, pathological or infectious response, in addition to its structural characteristics which are convenient for stabilizing metallic nanoparticles.

Chitosan is the name given to a polymer resulting from the deacetylation of chitin, a biodegradable natural compound derived from crustacean shells such as crabs, shrimps and whose main attribute is its polycationic nature.

Chitosan is mainly composed of glucosamine, 2-amino-2-deoxy-bD-glucose which gives the polymer numerous physiological and biological unique properties, with great potential in a wide range of industries, such as cosmetics and other products for:
personal care: as lotions, facial additives, hair products and body creams;
food: industrialized as coating, preservative, antioxidant, antimicrobial;
biotechnology: as chelator, emulsifier, flocculant;
pharmacology: and medicine as fibers, fabrics, drugs, membranes, artificial organs;
agriculture: as a soil modifier, films, fungicide, elicitors.

Chitosan and silver nanoparticles are two materials with antimicrobial properties already demonstrated. Some authors have shown synergy in inhibiting the in vitro growth of the methicillin-resistant Gram-positive Staphylococcus aureus (MRSA) and Gram-negative bacteria: Pseudomonas aeruginosa, Proteus mirabilis, Acinetobacter baumannii.

It is also known that fluorine has antibacterial properties and the ability to remineralize tooth tissues.

Specifically in the dental field, some documents mention materials containing silver particles of nanometric dimensions for use in restorations: The WO 2012003290 document describes nanometric calcium phosphate dental composites and the WO 2011049719 document presents kits for ceramic coatings for dental applications.

Other documents describe the combination of silver with polysaccharides to form antimicrobial compositions. For example, the WO 2006/026026 document presents a composition formed by solvent, silver nanoparticles and a stabilizing agent, which can be used for oral and dental hygiene products. Similarly, the US 2008/0147019 document and the US 2011/0129536 document describe the combination of nanometric silver stabilized with polysaccharides as, for example, chitosan and its derivatives.

The U.S. Pat. No. 6,565,873 document describes a biodegradable controlled release system containing nanoparticles, suitable for oral cavity and other mucous membranes and susceptible to be incorporated in dentifrices and mouthwashes formulations. The U.S. Pat. No. 6,790,460 document describes similar compositions, more specifically for the mucous membrane and the oral cavity.

Other documents, such as U.S. Pat. No. 7,713,955, report methods and systems for surface coating, based on polycyclic systems and quaternary ammonium compounds in which some compositions may contain silver.

The U.S. Pat. No. 8,119,162 document describes particles that disintegrate or prevent bacterial adhesion to oral hygiene and compositions containing particles connected to bio adhesive polymers for inhibiting the formation of films, bacterial plaque, biofilms and bio corrosion. The oral compositions described herein indicate the use of bio adhesive polymers combined with inorganic particles.

The U.S. Pat. No. 8,241,651 document describes multiphase, bio functional nanocomposites for use in medical diagnostic, pharmaceutical, personal care, oral hygiene and nutrition methods, enabling the controlled release of its components or the combination of incompatible active ingredients. Some of the mentioned applications are antimicrobial coatings, anticorrosive, sensors for bio image with combined use for diagnosis and treatment. These systems can be formed through electro spinning.

The U.S. Pat. No. 8,252,851 document describes polymerizable reagents which may be used as materials for tooth restoration with good mechanical properties. They can be used as dental compounds, bone implants, and may also be used for releasing active ingredients as DNA, fluoride or antibacterial agents.

As seen in the above report, the silver diammine fluoride (SDF) which is now topically used in the prevention and interruption of dental caries has two major limitations: (i) the carious region is excessively dark, which often leads to the rejection of its use by patients and dentists; (Ii) it is an aqueous solution of low viscosity and high concentration of silver ions. The low viscosity hinders the application on the affected area and the product often flows into unwanted areas, including on the skin of the professional who makes the application. The affected areas become dark after a few days and the stains formed in the skin usually last for a few weeks.

In contrast, despite the significant amount of information in the scientific literature and documents claiming various applications of systems containing ionic silver or silver particles of nanometric dimensions as an antimicrobial agent and more specifically antibacterial, bacteriostatic, or bactericidal, including the dental field, it is observed that the technique resents itself from a product that leads to a combination of prevention, treatment, remineralization in a single product, and from the combination of antimicrobial properties with optical properties of particles with nanometric dimensions.

### SUMMARY OF THE INVENTION

The objectives of the invention are to provide a product designed to prevent the occurrence of lesions in teeth caused by dental caries, interrupt the development of such lesions, as well as promote the remineralization of the affected tooth, as well as a process for obtaining such product.

The first object is achieved through a hybrid complex nanostructured product that comprises: metal particles with dimensions ranging between 2 and 500 nanometers and/or free or complexed metal ions; fluoride; and, eventually, organic acid and polysaccharides, with or without additives which can be present in its liquid form, with variable viscosity, in the form of moldable mass or even of self-supporting film, whose color depends on the optical properties of metal particles, their interactions with the dispersing medium and with other components of the formulation.

The second objective is achieved through a process for obtaining this product and which comprises the following basic steps: dissolving a compound containing a metal ion to obtain a first solution; preparing a second solution containing a reducing agent; dissolving a polysaccharide in water or in an aqueous organic acid solution to obtain a third solution; mixing all three solutions in order to generate metal nanoparticles; to submit solutions as well as their mixtures to temperature effects; adding solid fluoride salt or in aqueous solution to the mixture; and add a stabilizing agent.

### DETAILED DESCRIPTION OF THE INVENTION

This invention is about a product designed to prevent the occurrence of lesions in teeth caused by dental caries, interrupt the development of such lesions, as well as promote the remineralization of the affected tooth and a process of obtaining this product.

The product gives the affected area a visually identifiable color, which enables, in some cases, monitoring evolution of the treatment over time.

The final product may be presented in liquid, mass or film form and, by the properties conferred by its components, it promotes an antimicrobial action, particularly bacteriostatic and bactericidal, with the capacity to prevent and stop the carious lesion together with the capacity to assist in teeth remineralization.

Once applied, the product gives the affected area a visually identifiable color, which enables, in some cases, monitoring the evolution of the treatment over time. The product enables or increases access to dental treatment, as it does not require an outpatient setting for its application. As this form of treatment does not involve invasive techniques for its application, the product is particularly recommended for treating children or people who react negatively to traditional places of dental intervention.

In the case of the applications proposed herein, it is exploited, in addition to its bacteriostatic and bactericidal properties, the visual aspect of formulations containing metal nanoparticles which enable the monitoring of the prophylaxis result, identifying more accurately the affected areas, with favorable aesthetic appearance.

In some cases, with time after the application of a varnish, the product presents visually identifiable color changes. This happens because the colloidal dispersions that contain metal particles of nanometric dimensions have various colors depending on the size distribution, shape, interaction with passivating/stabilizers molecules and the environment in which they are dispersed. This is a typical characteristic of the nanometric regime that results from oscillation of the so-called surface plasmons.

The composition of the product, one of the objectives of this invention, comprises the following components described below:
0.001% to 0.50% in metal particles selected among silver, gold, copper, gold/silver alloys and gold/copper alloys, present several morphologies such as spherical, elliptical, prisms, rods and plates, with dimension values ranging from 1 nm to 500 nm, are obtained from precursor compounds of metals through controlled reduction reactions, where the precursor compounds are selected among oxides, acetates, nitrates, acetyl acetonates, carbonate, chloride, citrate, fluoride, iodate, lactate, iodide, nitrite, perchlorate, phosphate, sulfate and trifluoroacetate;
0.03% to 6.0% of a polysaccharide chitosan with molar mass ranging between 20 kDa and 10,000 kDa, its derivatives, which are selected among: carboxymethyl chitosan, chitosan lactate, acetate chitosan, amylopectin, amylose, alginates, alginic acid, pectin, xylan, and chitin, other biopolymers selected from: methyl cellulose, carboxymethyl cellulose, cellulose acetate, hydroxyethyl cellulose and hydroxypropyl cellulose, and, additionally, salts deriving from alginic acid sodium, potassium or calcium;
0.05% to 2.0% of a fluoride salt, which tends to be associated with cationic sites of the polysaccharide obtained from water-soluble salts, from aqueous solutions with an acidic pH up to a maximum value of 6.0 in both stock solutions;
0.05% to 4.0% of an organic acid selected among lactic acid, acetic acid, citric acid, oxalic acid, ascorbic acid and other organic acids of general RCOOH structure, where R refers to an organic radical;
0.01% to 2.0% of a liquid dispersing agent based on water, with a slightly acid pH eventually increased with co-solvent, which is chosen among glycols such as propylene glycol, poly (ethylene glycol), including different molar masses, substituted glycols, co-solvents such as ethanol, isopropanol and polysorbates and other polymeric additives for controlling the viscosity derived from cellulose as hydroxy-propyl, methyl cellulose, hydroxy-propyl cellulose, carboxy methyl cellulose and methyl cellulose;
0.01% to 10% of a flavoring chosen among: peppermint, mint, cinnamon, eucalyptus, cherry, strawberry and red berries.

The metal particle is preferably the silver, and the stoichiometric ratio of metal ion to reducing agent ranges from 0.10 to 10.0.

The different forms of the product with its individual component options include the addition of medium or long chain polysaccharides that perform the following functions:
antibacterial activity;
increased physical and chemical stability for the compositions, which results in an increased expiration time for products;
Stabilization of the nanoparticles resulting from interactions between the surface of the particles and functional groups and/or loads located in the structure of polysaccharides;
ionic association with ions present in the composition and the ability to obtain compositions with higher concentration of active principle.

The cationic character of chitosan and the anionic character of fluoride enables the property to form associations of “fluorides chitosan” type and soluble complexes of non-stoichiometric fluoride chitosan and when chitosan is adsorbed on silver nanoparticles, complex and hybrid particles are formed.

Some polysaccharides can be dissolved with any heating up to 50° C. or directly in water, without the necessity of adding any organic acid.

The presentation of the final product may be chosen among:
A viscous liquid whose color depends on the optical properties of the particles of nanometric dimensions and their interactions with the environment in which it is dispersed. It is already known by experts that the optical properties of the particles of nanometric dimensions depend on its size, geometry and size distribution and geometric shape for each component option of the composition. For example, a composition containing gold may be yellow or brownish, while another composition containing gold may also be red, blue or purple, depending on the other components involved;
Solid films, which are directly obtained from liquid formulations through controlled evaporation of the solvent, followed or not by reticulation with alkaline vapors;
A material in form of paste, obtained from wet films;
A material in form of mass, which is obtained from wet films, which are folded and pressed until they acquire an aspect similar to a moldable mass.

Regardless the final product presentation, the application can be: topical on the surface of healthy teeth for prevention, as well as in cavities and tissues affected by carious lesion for treating and remineralization.

The process for obtaining the product and to prevent the interruption of carious lesion and teeth remineralization, the second object of the present invention, will be described below and can be observed that it comprises the following steps:
Preparing a first aqueous solution containing metal ions concentrated in values ranging from 0.010 to 0.33 moles per liter;
Preparing a second aqueous solution containing a reducing agent concentrated in values ranging from 0.015 to 1.250 moles per liter, which is chosen among hydrazine, sodium citrate, sodium borohydride, glucose, ascorbic acid, albumin, LiAlH4, boranes and some polysaccharides which may also perform this function;
Preparing a third solution chosen among:
   dissolving a polysaccharide whose molar mass must be in a range of values between 20 and 10000 kDa in water; and
   dissolving a polysaccharide in an aqueous solution of an organic acid from 0.5 to 5.5%, adding 0.05 grams to 5.0 volumes of up to 500 mL, depending on the solubility of the polysaccharide;
Submit solutions to temperature effects on a range of values between 3° C. to 60° C.;
Mix the solutions to generate metal nanoparticles and whose resulting form is chosen among:
   adding the first solution to the third solution and then adding the second solution to the mixture;
   adding the second solution to the first solution and then adding the third solution;
Adding a fluoride salt to the mixture of solutions in a form chosen among: dissolving the solid salt directly in the mixture and adding an aqueous solution of fluoride salt to the mixture;
Adding a flavoring agent.

The final product obtained through the process above, through its characteristics, has the following advantages:
greater efficiency for the prevention and eradication of dental caries in comparison to currently used products and with a similar application method;
the silver content may be up to 17 times lower than the silver diammine fluoride commercially used in products;
metal nanoparticles provide an acceptable visual appearance, inexistent in comparable technologies. The total content of silver is up to 600 times smaller than in the SDF to obtain the same clinical effect;
overall, the evolution of the treatment can be monitored over time, from visual inspection and color identification of the compromised areas indicating, also, the necessity for new applications;
the high viscosity of the end product prevents the product flowing to unwanted areas, and avoids the subsequent formation of spots on the skin of the patient or professional;
low cost compared to treatments carried out in offices;
the product does not require a dental clinic equipped for application and does not require the use of drills or procedures requiring anesthesia, because its application is painless;
simple application, which can be performed with brush or through direct application of membrane or mass.

Some examples involving preparation aspects in some of the stages of the process for obtaining the final product and results observations will be presented below.

### EXAMPLE 1

Addresses the chemical reduction of silver ions with sodium citrate, using AgNO3 as a precursor, where 90 mg of AgNO3 were dissolved in 500 mL of deionized water and the solution was heated to boiling under magnetic stirring. Then, 10 mL of sodium citrate solution at 1% (w/v) were added. The solution was kept under stirring and boiled for 1 hour. The nanoparticles synthesized by this method presented an absorption maximum at approximately 420 nm in the UV-visible absorption spectrum.

### EXAMPLE 2

100 ml of an aqueous ascorbic acid solution (6×10−4 mol×L−1) were used, sodium citrate (3×10−3 M) and the pH was controlled in the range 6.0 to 10.5 by adding 0.2 M of HNO3 or 0.1 M of NaOH. The system was heated to 35° C. under magnetic stirring and 1 ml of a 0.10 M solution of AgNO3 was added. The solution turned brown for the synthesis with pH =10.5 and blurred blue for those with pH <10.5.

### EXAMPLE 3

For the synthesis of seeds, in a solution 4×10−4 M of AgNO3, under magnetic stirring and room temperature, 1 mL of 0.072 M of NaBH4 was quickly added, immediately turning the solution yellow after its addition. Thus, many nuclei (seeds) were generated, which were stabilized by chitosan and served for the growth of larger nanoparticles. For the growth of the silver nanoparticles (AGNPS), initially in 50 mL of a 4.0×10−4 M of AgNO3 solution and 20.0×10−4 M of sodium citrate, under magnetic stirring and room temperature, different amounts of seed (10.0μL−3.0mL) and 1.0 mL of a 2.0×10−3 M solution of ascorbic acid were added. In this case, the addition of ascorbic acid had to be carried out slowly and the final size and shape of the AgNPs at the end of the synthesis depended on the amount of seeds, or in other words, small quantities of seeds result in higher AgNPs and large quantities of seeds results in lower AgNPs.

### EXAMPLE 4

To prepare the colloid, 1.0 gram of chitosan was dissolved in 200 ml of acetic acid solution at 2% (v/v). The solution was kept overnight under stirring and then the solution was vacuum filtered. It was later taken one aliquot of 60 mL of chitosan solution and placed in an ice bath under stirring. Then, 4.0 mL of a 0.012 mol×L−1 silver nitrate solution were added and 30 more minutes were included before adding sodium borohydride. The relationship between AgNO3 and NaBH4 was maintained 1:6 in mass with dropwise addition. The reduction of Ag+ was started immediately, the color of the solution changed from colorless to light yellow and finally red. After synthesis, the colloid remained in the ice bath for 45 minutes. After this period, the colloid was taken out of the ice bath colloid and we waited for it to reach the room temperature before the optical absorption measurements. Immediately after the synthesis, the samples were stored in vials protected with aluminum foil and were and kept in the refrigerator.

Although the present invention has been described in its preferred embodiment, the main ideas guiding the present invention which are a product designed to prevent the occurrence of lesions in teeth caused by dental caries, interrupt the development of such lesions, as well as promote remineralization of the affected tooth and a process for obtaining this product remains preserved as to its innovative character, where those usually skilled in the technique may discern and practice variations, modifications, alterations, adaptations and equivalents that are suitable and compatible to the working environment in question without, however, departing from the comprehensiveness of the spirit and scope of the invention, that are represented by the following claims.

1. PRODUCT FOR PREVENTION, INTERRUPTION OF CARIOUS LESION AND REMINERALIZATION OF TEETH characterized by comprehending the following components:
0.001% to 0.50% in metal particles selected among silver, gold, copper, gold/silver alloys and gold/copper alloys, present several morphologies such as spherical, elliptical, prisms, rods and plates, with dimension values ranging from 1 nm to 500 nm, are obtained from precursor compounds of metals through controlled reduction reactions, where the precursor compounds are selected among oxides, acetates, nitrates, acetyl acetonates, carbonate, chloride, citrate, fluoride, iodate, lactate, iodide, nitrite, perchlorate, phosphate, sulfate and trifluoroacetate;
0.03% to 6.0% of a polysaccharide chitosan with molar mass ranging between 20 kDa and 10,000 kDa, its derivatives, which are selected among: carboxymethyl chitosan, chitosan lactate, acetate chitosan, amylopectin, amylose, alginates, alginic acid, pectin, xylan, and chitin, other biopolymers selected from: methyl cellulose, carboxymethyl cellulose, cellulose acetate, hydroxyethyl cellulose and hydroxypropyl cellulose, and, additionally, salts deriving from alginic acid sodium, potassium or calcium;
0.05% to 2.0% of a fluoride salt, which tends to be associated with cationic sites of the polysaccharide obtained from water-soluble salts, from aqueous solutions with an acidic pH up to a maximum value of 6.0 in both stock solutions;
0.05% to 4.0% of an organic acid selected among lactic acid, acetic acid, citric acid, oxalic acid, ascorbic acid and other organic acids of general RCOOH structure, where R refers to an organic radical;
0.01% to 2.0% of a liquid dispersing agent based on water, with a slightly acid pH eventually increased with co-solvent, which is chosen among glycols such as propylene glycol, poly (ethylene glycol), including different molar masses, substituted glycols, co-solvents such as ethanol, isopropanol and polysorbates and other polymeric additives for controlling the viscosity derived from cellulose as hydroxy-propyl, methyl cellulose, hydroxy-propyl cellulose, carboxy methyl cellulose and methyl cellulose;
0.01% to 10% of a flavoring chosen among: peppermint, mint, cinnamon, eucalyptus, cherry, strawberry and red berries.

2. PRODUCT FOR PREVENTION, INTERRUPTION OF CARIOUS LESION AND TEETH REMINERALIZATION according to claim 1, characterized by the metal particle being preferably silver, and the stoichiometric ratio of metal ion to reducing agent ranges from 0.10 to 10.0.

3. PRODUCT FOR PREVENTION, INTERRUPTION OF CARIOUS LESION AND TEETH REMINERALIZATION according to claim 1, characterized by the different forms of the product with its individual component options including the addition of medium or long chain polysaccharides that perform the following functions:
antibacterial activity;
increased physical and chemical stability for the compositions, which results in an increased expiration time for products;
stabilization of the nanoparticles resulting from interactions between the surface of the particles and functional groups and/or loads located in the structure of polysaccharides;
ionic association with ions present in the composition and the ability to obtain compositions with higher concentration of active principle.

4. PRODUCT FOR PREVENTION, INTERRUPTION OF CARIOUS LESION AND TEETH REMINERALIZATION according to claim 1 characterized by the cationic character of chitosan and the anionic character of fluoride enabling the property to form associations of “fluorides chitosan” type and soluble complexes of non-stoichiometric fluoride chitosan and the formation of complex and hybrid particles when chitosan is adsorbed on silver nanoparticles

5. PRODUCT FOR PREVENTION, INTERRUPTION OF CARIOUS LESION AND TEETH REMINERALIZATION according to claim 1 characterized by some polysaccharides being able to be dissolved with any heating up to 50° C. or directly in water, without the necessity of adding any organic acid.

6. PRODUCT FOR PREVENTION, INTERRUPTION OF CARIOUS LESION AND TEETH REMINERALIZATION according to claim 1 characterized by the presentation of the final product being able to be chosen among:
A viscous liquid whose color depends on the optical properties of the particles of nanometric dimensions and their interactions with the environment in which it is dispersed.
Solid films, which are directly obtained from liquid formulations through controlled evaporation of the solvent, followed or not by reticulation with alkaline vapors;
A material in form of paste, obtained from wet films;
A material in form of mass, which is obtained from wet films, which are folded and pressed until they acquire an aspect similar to a moldable mass.

7. PRODUCT FOR PREVENTION, INTERRUPTION OF CARIOUS LESION AND TEETH REMINERALIZATION according to claim 1 characterized by regardless the final product presentation, the application being able to be: topical on the surface of healthy teeth for prevention, as well as in cavities and tissues affected by carious lesion for treating and remineralization.

8. PROCESS FOR OBTAINING THE PRODUCT characterized by comprehending the following steps:
Preparing a first aqueous solution containing metal ions concentrated in values ranging from 0.010 to 0.33 moles per liter;
Preparing a second aqueous solution containing a reducing agent concentrated in values ranging from 0.015 to 1.250 moles per liter, which is chosen among hydrazine, sodium citrate, sodium borohydride, glucose, ascorbic acid, albumin, LiAlH4, boranes and some polysaccharides which may also perform this function;
Preparing a third solution chosen among:
dissolving a polysaccharide whose molar mass must be in a range of values between 20 and 10000 kDa in water; and
dissolving a polysaccharide in an aqueous solution of an organic acid from 0.5 to 5.5%, adding 0.05 grams to 5.0 volumes of up to 500 mL, depending on the solubility of the polysaccharide;
Submit solutions to temperature effects on a range of values between 3° C. to 60° C.;
Mix the solutions to generate metal nanoparticles and whose resulting form is chosen among:
adding the first solution to the third solution and then adding the second solution to the mixture;
adding the second solution to the first solution and then adding the third solution;
Adding a fluoride salt to the mixture of solutions in a form chosen among: dissolving the solid salt directly in the mixture and adding an aqueous solution of fluoride salt to the mixture;
Adding a flavoring agent.
